(19) United States
(12) Patent Application Publication
(10) Pub. No.: US 2013/0095171 A1
(43) Pub. Date: Apr. 18, 2013

(54) HERBAL COMPOSITION AND METHOD FOR TREATMENT OF AIRWAY INFLAMMATION USING THE SAME

(71) Applicant: Jaw-Ji Tsai, Taipei City (TW)

(72) Inventor: Jaw-Ji Tsai, Taipei City (TW)

(73) Assignee: Taichung Veterans General Hospital, Taichung City (TW)

(21) Appl. No.: 13/649,460

(22) Filed: Oct. 11, 2012

Related U.S. Application Data
(60) Provisional application No. 61/546,649, filed on Oct. 13, 2011.

(51) Int. Cl. A61K 36/8945; A61P 37/08; A61P 11/06; A61P 11/02; A61K 9/127; A61P 29/00

(52) U.S. Cl. 424/450; 424/728

(57) ABSTRACT

An herbal composition includes Dioscorea opposita, Nelumbo nucifera seeds, Euryale ferox seeds, Poria cocos, Diospyros kaki, Prunus dulcis, Mentha piperita leaves, and Panax quinquefolius. A method for treatment of airway inflammation in a mammal includes administering to the mammal in need of such treatment the aforesaid herbal composition.

### CROSS-REFERENCE TO RELATED APPLICATION

This application claims priority of U.S. Provisional Patent Application No. 61/546,649, filed on Oct. 13, 2011.

### BACKGROUND OF THE INVENTION

1. Field of the Invention

This invention relates to an herbal composition for treatment of airway inflammation, more particularly to an herbal composition for treatment of allergen-induced airway inflammation.

2. Description of the Related Art

Allergen-induced airway inflammation, e.g., allergic rhinitis, allergic conjunctivitis, and allergic bronchial asthma, is one of the most common allergic diseases in developed countries. According to American Lung Association and WHO, 34 million Americans and 300 million people worldwide suffer from asthma. Allergic asthma is characterized by airway hyper-responsiveness and airway inflammation (A. B. Kay, Asthma and inflammation, J. Allergy Clin. Immunol. 1991; 87:893-910), which results in symptomatic effects including, but not limited to, episodic breathlessness, wheezing, chest tightness, and cough that worsen at night and in the early morning (R. F. Lemanske, A review of the current guidelines for allergic rhinitis and asthma. J. Allergy Clin. Immunol. 1998; 101:S392-S396). These symptoms are associated with elevated levels of serum allergen-specific IgE and IL-4 released from allergen specific CD 4 cells expressing the Th2 cytokine profile. Since Th1 cytokines may down regulate Th2 function to reduce IgE, the hypothesis that a shift in polarization of cytokine production from a Th2 to a Th1 cytokine profile has been proposed to provide more specific therapeutic means for allergic asthma.

Dexamethasone is a potent synthetic member of the glucocorticoid class of steroid drugs. It acts as an immunosuppressant, and is commonly used as a drug for asthma treatment. However, due to its undesired side effects, i.e., obesity, reduced immunity, osteoporosis, cataract, etc., many approaches have been investigated to treat allergic asthma. Treatment using Traditional Chinese Medicines (TCMs) is one of these approaches.

Fritillaria unibracteata is a well-known and precious Traditional Chinese Medicine, and belongs to the family of Liliaceae. In Chinese herbal practices, Fritillaria unibracteata is effective for relieving cough, removing phlegm, and reducing fever and airway inflammation.

Dioscorea opposita, Nelumbo nucifera seeds, Euryale ferox seeds, and Poria cocos are commonly used together to stimulate spleen function and increase appetite.

Diospyros kaki and Prunus dulcis are both used to treat coughing and wheezing.

Mentha piperita, commonly referred to as peppermint, is widely used to treat coughing and reduce phlegm.

Schizonepeta tenuifolia is a well-documented anti-allergy and anti-inflammation herb in TCM.

Panax quinquefolius, also known as American ginseng, is noted for its anti-inflammatory effect via anti-apoptosis. In TCM, Panax quinquefolius is also believed to exhibit effect of promoting overall sense of well-being and boosting immune system.

Some of the aforesaid herbs have been found to be effective to relieve asthma symptoms. One study has shown that Fritillaria modulates airway inflammation by suppression of cytokines, IgE, histamine production, and eosinophilic accumulation along with increased IFN-γ production in lung tissue (H. S. Yeum et al., Basic Clin. Pharmacol., 2007; 100:205-213). An extract form Mentha piperita is reported to be effective in alleviating nasal symptoms of allergic rhinitis due to its ability to inhibit antigen-induced histamine release (T. Inoue et al., Biol. Pharm. Bull., 2002; 25:256-259).

Although some of the aforesaid herbs can be used to relieve asthma symptoms, in practical use, effect of combination use of the herbs in treatment of the airway inflammation is unknown. Moreover, ratio of the herbs is likely to influence the effect thereof. Therefore, for a herb complex, herb components and ratio thereof play important roles in treating effectiveness of the herb complex. In addition, since Chinese herbs are usually orally administered, the effectiveness thereof is also determined by absorption thereof in gastrointestinal tract.

Dioscorea opposite, also known as Chinese wild yam, is a common herb in Asia. Its dried form or powder form is used frequently in TCM for improving spleen, lung and kidney functions. Dioscorea opposite is reported to be useful for treating diarrhea, chronic loose stool and poor appetite.

Nelumbo nucifera seeds have been reported to exhibit functions of lowering cholesterol levels and relaxing smooth muscle of the uterus. They have been used for poor digestion, enteritis (inflammation of small intestine), chronic diarrhea, and palpitations.

Euryale ferox seeds are commonly used in TCM to cure a variety of diseases including kidney problems, chronic diarrhea, excessive leucorrhea and hypofunction of the spleen.

Poria cocos, also known as Fu Ling, is a fungus widely used in TCM to enhance lung function.

In this invention, considering absorption of Chinese herbs and the spleen function in immune system, the applicant of this invention has found that combination of Dioscorea opposita, Nelumbo nucifera seeds, Euryale ferox seeds, Poria cocos, Diospyros kaki, Prunus dulcis, Mentha piperita leaves, and Panax quinquefolius can be effectively used to treat airway inflammation. Fritillaria unibracteata and Schizonepeta tenuifolia can also be used with the aforesaid combination.

### SUMMARY OF THE INVENTION

The object of the present invention is to provide a novel Chinese herbal composition for treatment of airway inflammation.

According to one aspect of this invention, an herbal composition includes Dioscorea opposita, Nelumbo nucifera seeds, Euryale ferox seeds, Poria cocos, Diospyros kaki, Prunus dulcis, Mentha piperita leaves, and Panax quinquefolius.

According to another aspect of this invention, a method for treating airway inflammation in a mammal includes administering to the mammal in need of such treatment an herbal composition, the herbal composition including, Dioscorea opposita, Nelumbo nucifera seeds, Euryale ferox seeds, Poria cocos, Diospyros kaki, Prunus dulcis, Mentha piperita leaves, and Panax quinquefolius.

### BRIEF DESCRIPTION OF THE DRAWINGS

Other features and advantages of the present invention will become apparent in the following detailed description of the preferred embodiments of this invention, with reference to the accompanying drawings, in which:

FIG. 1 shows index of Penh for mice in groups 1, 2, 3, 4, and 5 at different concentrations of methacholine aerosol, in which mice in group 1 were not immunized with a DP emulsion and were not treated with a drug, mice in group 2 were immunized with a DP emulsion but were not treated with any drug, mice in group 3 were immunized with a DP emulsion and treated with Dexamethasone, and mice in group 4 were immunized with a DP emulsion and treated with an herbal composition of this invention;

FIG. 2 shows IgG1 levels in sera for groups 1 to 5;

FIG. 3 shows IgE levels in sera for groups 1 to 5;

FIG. 4 shows IL-4 concentration in sera for groups 1 to 5;

FIG. 5 shows IFN-γ concentration in sera for groups 1 to 5;

FIG. 6 shows the histological scores for groups 1 to 5; and

FIG. 7 shows photographs of lung tissues for groups 1 to 5.

### DETAILED DESCRIPTION OF THE PREFERRED EMBODIMENTS

This invention provides an herbal composition for treatment of airway inflammation in a mammal. The herbal composition of the present invention comprises Dioscorea opposita, Nelumbo nucifera seeds, Euryale ferox seeds, Poria cocos, Diospyros kaki, Prunus dulcis, Mentha piperita leaves, and Panax quinquefolius.

Preferably, in the herbal composition of this invention, Dioscorea opposita, Nelumbo nucifera seeds, Euryale ferox seeds, Poria cocos, Diospyros kaki, Prunus dulcis, Mentha piperita leaves, and Panax quinquefolius are in a ratio of 1:1:1:1:4:4:4:10 by weight.

Preferably, the herbal composition of this invention further includes Fritillaria unibracteata and Schizonepeta tenuifolia, and Fritillaria unibracteata, Dioscorea opposita, Nelumbo nucifera seeds, Euryale ferox seeds, Poria cocos, Diospyros kaki, Prunus dulcis, Mentha piperita leaves, Schizonepeta tenuifolia and Panax quinquefolius are in a ratio of 20:1:1:1:1:4:4:4:4:10 by weight.

Optionally, the herbal composition of this invention can comprise a pharmaceutically acceptable carrier. For instance, the pharmaceutically acceptable carrier may include one or more of the following agents: solvents (e.g., water, normal saline, buffer solution, glycerin, organic solvents), emulsifiers, suspending agents, disintegrators, binders, excipients, preservatives, lubricants, absorption delaying agents, liposomes, coloring agents, flavoring agents, stabilizers, and the like.

This invention also provides a method for treatment of airway inflammation in a mammal. The method includes administering to the mammal in need of such treatment the herbal composition of this invention.

The airway inflammation of this invention includes allergen-induced airway inflammation. The “allergen-induced airway inflammation” used herein comprises, but is not limited to, allergic rhinitis, allergic conjunctivitis, or allergic bronchial asthma. Allergens that might induce the allergen-induced airway inflammation include, but are not limited to, dust mite, pollen, mold, animal dander, cockroach, food, or combinations thereof.

The herbal composition of this invention is preferably orally administered.

The herbal composition of this invention can be formulated into a suitable dosage form, which includes, but is not limited to, sterile aqueous solutions, dispersions, sterile powder, tablets, lozenges, pills, capsules, caplets, and the like.

The dosage and the frequency of administration of the herbal composition according to this invention will vary depending on the following factors: the severity of the disease to be treated, the route of administration, and the weight, age, physical condition and response of the subject to be treated. For instance, the daily dosage of the herbal composition for a mouse according to this invention may be 0.1 mg/kg/day to about 300 mg/kg/day, and may be administered in a single dose or in several doses.

As described in Examples below, the present invention demonstrates successful use of the herbal composition of this invention to treat allergen-induced airway inflammation.

### EXAMPLE

### Materials and Methods

### 1. Animals

Male Balb/c mice (6 to 8 weeks of age) were obtained from the National Laboratory Breeding Research Center in Taiwan and were kept in a specific pathogen-free environment with 12 hr dark/12 hr light cycle, temperatures of 21-22° C., and 30-70% humidity. Diet and water were sufficient and accessible to the mice at all times. All animal experiments were conducted according to Guides for the Care and Use of Laboratory Animals of National Institute of Health (NIH).

2. Preparation of the Herbal Composition of this Invention

Herbs used in this invention were purchased from Shiun Tien Tang Co. Ltd., (Taipei, Taiwan). Herbal composition TCM-A was obtained by mixing Fritillaria unibracteata, Dioscorea opposita, Nelumbo nucifera seeds, Euryale ferox seeds, Poria cocos, Diospyros kaki, Prunus dulcis, Mentha piperita leaves, Schizonepeta tenuifolia and Panax quinquefolius at a ratio of 20:1:1:1:1:4:4:4:4:10 by weight. Herbal composition TCM-B was obtained by mixing Dioscorea opposita, Nelumbo nucifera seeds, Euryale ferox seeds, Poria cocos, Diospyros kaki, Prunus dulcis, Mentha piperita leaves, and Panax quinquefolius at a ratio of 1:1:1:1:4:4:4:10 by weight.

### 3. Preparation of Der-p2 Allergen

Der-p2 gene (NCBI gene ID No.: EU 346693) was transformed into Pichia host strain GS115 using a Pichia protein expression kit (Invitrogen, Catalog no: K1710-01) followed by cultivating in YPD medium at 28-30° C. for 2 days. The cells were collected and then were cultivated in a BMGY medium (pH was adjusted to 7.0 using HCl and NaOH) in a 300 ml flash at 28° C. and 150 rpm for 24 hours. Thereafter, dissolved oxygen (DO) content and pH value of the culture medium were detected. When DO content increased 10% and pH value decreased 10%, 50% glycerol in water was added into the BMGY medium at a rate of 4.5 ml/h for 5 hours and the dissolved oxygen (DO) content was measured. When the DO content increased 10%, methanol (10% by volume of the total volume of the fluid in the flask) was added into the BMGY medium once a day for 4 days, followed by centrifugation to obtain a supernatant. Thereafter, the supernatant were collected and added with ammonium sulfate (50% by volume of the volume of the supernatant), followed by standing at 4° C. for 2 days. Centrifugation at 10000 rpm was then conducted and a pellet was obtained. The pellet was dialyzed in a PBS buffer for 2 days, followed by lyophilisation so as to obtain Der-p2 allergen.

### 4. Induction of Allergic Airway Inflammation

Dermatophagoides pteronyssinus was subjected to ultrasonically vibrating treatment so as to obtain Dermatophagoides pteronyssinus (DP) powder. 50 μg DP powder was dissolved in 150 μL PBS and was emulsified with 150 μL Al (OH)₃ (13 mg/ml), Whitehall Lab Ltd, Punchbowel, Australia) to obtain a DP emulsion. Mice were immunized by intraperitoneal (IP) injection with the DP emulsion on day 0 and day 7. The herbal composition was suspended in water to obtain a herbal suspension. The mice were administered orally with the herbal suspension of this invention (10 mg of herbal composition/300 μl of water/mouse/day) from day 21 to day 42. Administration was conducted once a day by injecting the herbal suspension to the esophagus of each mouse. On days 49 and 50, the mice were intratracheally (IT) inoculated with Der-p2 allergen (30 μg/30 μL PBS, obtained from the aforesaid “3. Preparation of Der-p2 allergen” in section of “Materials and Methods”). At day 51, airway hypersensitivity test was conducted. At day 58, the mice were sacrificed by CO₂.

### 5. Bronchoalveolar Lavage (BAL)

Bronchoalveolar lavage (BAL) was performed immediately after the mice were sacrificed at day 58 by the following procedure. 1 ml sterile endoxin-free saline fluid was injected into the lungs via trachea using a bronchoscope. 0.85 ml of the injected fluid (hereinafter referred to as a bronchoalveolar lavage fluid (BALF)) was recollected, followed by centrifugation at 200×g at 4° C. A pellet was used for differential BALF cell counts. BAL cells in the pellet were washed once with PBS. Thereafter, the cells were resuspended in RPMI-1640 (Sigma Chemical Co.). The total leukocyte count in BALF was determined using a hemocytometer. For differential BALF cell counts, cytospin preparations of 100 μl BALF were made and stained with Liu stain (Tonyar Diagnostic Inc, Taipei, Taiwain). Differential cell counts were performed on 200 cells and types of differentiated cells (i.e., leukocyte subpopulation) were observed by standard morphology.

### 6. Airway Hypersensitivity Test

Pulmonary function was measured by means of methacholine-induced airflow obstruction. Each mouse was placed inside a barometric plethysmograph (Buxco Electronics, Troy, N.Y., US). The plethysmograph has 2 chambers: one is the main or animal chamber (internal diameter, ID 7.5 cm and 5.5 cm height) and the other is the reference chamber (ID 7.5 cm and 3.5 cm height). A differential pressure transducer was employed to detect the pressure difference between the 2 chambers. The pressure signal was amplified, digitized via an A/D converter card, and sent to a computer with a BioSystem XA program (Buxco Electronics, Troy, N.Y., US), which sampled and calculated desired respiratory parameters including enhanced pause (Penh), tidal volume, breathing frequency, peak inspiratory flow, peak expiratory flow, end-inspiratory pause, and end-inspiratory pause.

An aerosol was generated by placing a 5 ml saline or methacholine solution in the cup of an ultrasonic nebulizer (DeVilbiss, Somerset, Pa., US) and was delivered via a connecting tube and a three-way connector to the animal chamber of the barometric plethysmograph. According to the manufacturer's information, the median amount of the aerosol was approximately 3 μm, and the range of the amount of the aerosol was from 1 to 5 μm. The aerosol usually filled the chamber within 15-20 sec. At first, each of the mice inhaled the saline aerosol for 3 minutes and then the respiratory parameters were measured for 3 minutes. Then, inhalation of the saline aerosol was replaced by the aerosolized methacholine in increasing concentrations (Sigma, 0 to 25 mg/ml, and each concentration was delivered through an inlet of the main chamber for 180 seconds. Then, the aerosol in the chamber was cleared immediately after the aerosol exposure, and the respiratory parameters were then measured for 3 minutes. The dose-response curve for the methacholine solution was plotted. There was a 15-min interval between each aerosol exposure. Parameters including respiratory rate, tidal volume, inspiratory time, expiratory time (Te, in seconds), peak inspiratory flow (Pif, in milliliters per second), peak expiratory flow (Pef, in milliliters per second), and relaxation time (Tr, in second) were recorded and averaged for 3 min after each nebulization, and enhanced pause (Penh) was determined based on these parameters. Penh reflects changes in pulmonary resistance during bronchoconstriction according to the following equation.

Penh=[(Te/0.3Tr)−1]×[2Pef/3Pif]

Baseline Penh measurement for each mouse was recorded for 3 minutes after each mouse was exposed to a saline aerosol. Penh index was obtained using the following equation:

Penh index=Penh[methacholine]/baseline Penh[saline].

### 7. Evaluation of Anti-Der-p2-Specific Antibody

Sera from vaccinated mice were collected at day 49. Anti-Derp 2-specific antibody (IgE and IgG1) levels were evaluated by ELISA. A 96-well plate (Maxisorp Nunc-Immuno plates, Life Technologies Inc.) was coated with 0.1 mL Der-p2 allergen (1 μg/well, obtained from the aforesaid “3. Preparation of Der-p2 allergen” in section of “Materials and Methods”) in a coating solution (14.3 mM Na₂CO₃, 10.3 mMNaHCO₃, pH 9.6), incubated at 4° C. overnight, and then was blocked with 1% BSA in PBS for 1 hour at room temperature. The serum samples were respectively added into the wells and were incubated for 2 hours at room temperature. Anti-mouse IgG1 and IgE biotinylated conjugates (purchased from Bethyl Laboratories) were respectively added into the wells to detect specific antibodies. After washing 8 times, plates were incubated at room temperature for 30 min with the Strept AB kit, and revealed by adding H₂O₂ with ortho-phenylenediamine. Color development was stopped by a stop solution of 2N H₂SO₄ and absorbance at 450 nm was measured.

8. Evaluation of IL-4 and IFN-γ in sera

Sera from vaccinated mice were collected at day 49. IL-4 and IFN-γ concentration were evaluated by ELISA. A 96-well plate (Maxisorp Nunc-Immuno plates, Life Technologies Inc.) was coated with IL-4 and INF-γ in a coating solution (14.3 mMNa₂CO₃, 10.3 mMNaHCO₃, pH 9.6), incubated at 4° C. overnight, and then was blocked with 1% BSA in PBS for 1 hour at room temperature. The serum samples were respectively added into the wells and were incubated for 2 hours at room temperature. Anti-mouse IL-4 and INF-γ (purchased from R&D Systems) were respectively added into the wells to detect specific antibodies. After washing 8 times, plates were incubated at room temperature for 30 min with the Strept AB kit, and revealed by adding H₂O₂ with ortho-phenylenediamine. Color development was stopped by a stop solution of 2N H₂SO₄ and absorbance at 450 nm was measured.

### 9. Histological Evaluation

After sacrifice, lung tissues were obtained and fixed in 10% formalin, followed by ethanol-dehydration and embedding in paraffin. The lung tissues were sectioned to obtain tissue sections, followed by mounting on slides coated with 3-aminopropyltriethoxy silane (APES). General morphology and cellular infiltration of the tissue sections were assessed using haematoxylin and eosin staining. Mucus-producing goblet cells were identified using periodic acid-Schiff (PAS) staining. The degree of cellular infiltration was scored using the method described by Kwon (Hsieh C W, Lan J L, Meng Q, Cheng Y W, Huang H M, Tsai J J. Eosinophil apoptosis induced by fungal immunomodulatory peptide-fve via reducing IL-5α receptor. J Formos Med Assoc. 2007 January; 106 (1):36-43). This scoring was determined based on the presence of mononuclear cells around blood vessels (score 0-3) and around bronchioli (score 0-3), and the number of patchy cellular infiltration (score 0-3). Histological scores were analyzed using non-parametric Mann-Whitney U test.

### EXAMPLE

Mice were randomly divided into five groups, with 6 mice in each group. Group 1 was not suffered from sensitization and was not treated with any drug. Groups 2, 3, 4, and 5 were sensitized with DP powder based on the procedures of the aforesaid “4. Induction of allergic airway inflammation” in section of “Materials and Methods”. At day 21, a blood sample was obtained via the orbital sinus of each mouse in groups 2, 3, 4, and 5, and IgG1, IgG2a, and IgE titers in serum of each mouse were measured using ELISA procedure set forth in “7. Evaluation of anti-Der-p2-specific antibody” in section of “Materials and Methods” to determination success of allergy induction. The mice with successful allergy induction were subjected to further experiment. Mice from Group 2 were left untreated, while mice from Groups 3, 4, and 5 were respectively treated with Dexamethasone, TCM-A and TCM-B prepared by the aforesaid “2. Preparation of the herbal composition of this invention” in section of “Materials and Methods”. The treatment of Dexamethasone and the herbal compositions was given daily from Day 21 to Day 42 after first immunization (day 0). The dose for Dexamethasone was 1 μg/mouse/day, and the dose for the herbal compositions was 10 mg of herbal composition/300 μl of water/mouse/day (i.e., about 500 mg/kg/day). On day 49, blood was obtained to evaluate anti-Derp 2-specific antibody and concentrations of IL-4 and IFN-γ in sera based on the aforesaid procedure of “7. Evaluation of anti-Derp 2-specific antibody” and “8. Evaluation of IL-4 and IFN-γ in sera” in section of “Materials and Methods”. On Day 51, the airway hypersensitivity test y in “Materials and Methods” was conducted to determine the degree of nasal obstruction, i.e., pulmonary function. Results for the airway hypersensitivity were expressed as an arithmetic mean±SEM. Differences between values before and after saline or methacholine exposure were analyzed by the paired t test. Differences between the saline control and the naïve groups were analyzed by the unpaired t test. Differences were considered significant if P<0.05.

After the mice were sacrificed, histological evaluation and bronchoalveolar lavage mentioned in section of “Materials and Methods” were performed.

### Results

### 1. Leukocyte Subpopulation in BALF

Leukocyte subpopulation in BALF of each group was analyzed and the results are shown in Table 1. It is well known that one of the objectives of asthma treatment is to eliminate or alleviate accumulation of eosinophils in lung. From Table 1, it is revealed that the herbal compositions, TCM-A and TCM-B, of this invention significantly reduced eosinophils in BALF (see Groups 4 and 5), indicating a relief of inflammatory symptoms and allergic eosinophilia.

TABLE 1

| | Macrophage | Lymphocyte | Neutrophil | Eosinophil |
|---|---|---|---|---|
| | (number of | (number of | (number of | (number of |
| Group | cell) | cell) | cell) | cell) |
| 1 | 295.6 ± 4.30 | 8.0 ± 0.10 | UD* | UD* |
| 2 | 98.5 ± 12.50 | 110.5 ± 10.20 | 190.5 ± 2.80 | 200.5 ± 1.20 |
| 3 | 110.0 ± 2.30 | 120.0 ± 5.70 | 98.0 ± 6.60 | 47.0 ± 3.00 |
| 4 | 110.3 ± 12.2 | 110.0 ± 2.10 | 130.5 ± 4.70 | 53.2 ± 8.90 |
| 5 | 135.0 ± 11.20 | 139.0 ± 2.90 | 150.2 ± 11.20 | 60.8 ± 3.90 |

*UD indicating un-detected.

### 2. Pulmonary Function

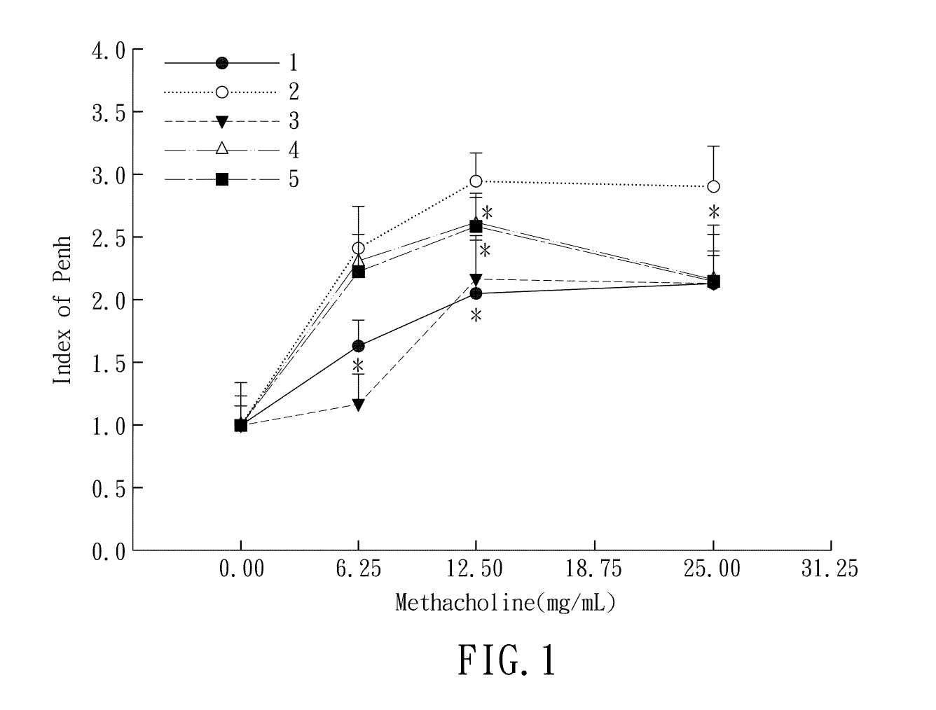

Pulmonary function was measured by airway hypersensitivity test on Day 51, and the results are shown in FIG. 1. The data reveals that the mice of groups 3, 4, and 5 have pulmonary function superior to that of group 2. The results show that there is a significant improvement in groups 3, 4, and 5, compared to the untreated group (group 2). Also, the data shows that the effect of the herbal composition of this invention is similar to that of dexamethasone, a common anti-inflammatory drug.

### 3. Antibody Evaluation

Allergic asthma is thought to be caused by an over Th2 response. IgG1 and IgE are associated with Th2 response, and decrease in IgG1 and IgE levels indicates less Th2 response. Thus, expression levels of IgG1 and IgE might be used to determine the effect of the herbal composition of this invention on treatment of allergic asthma.

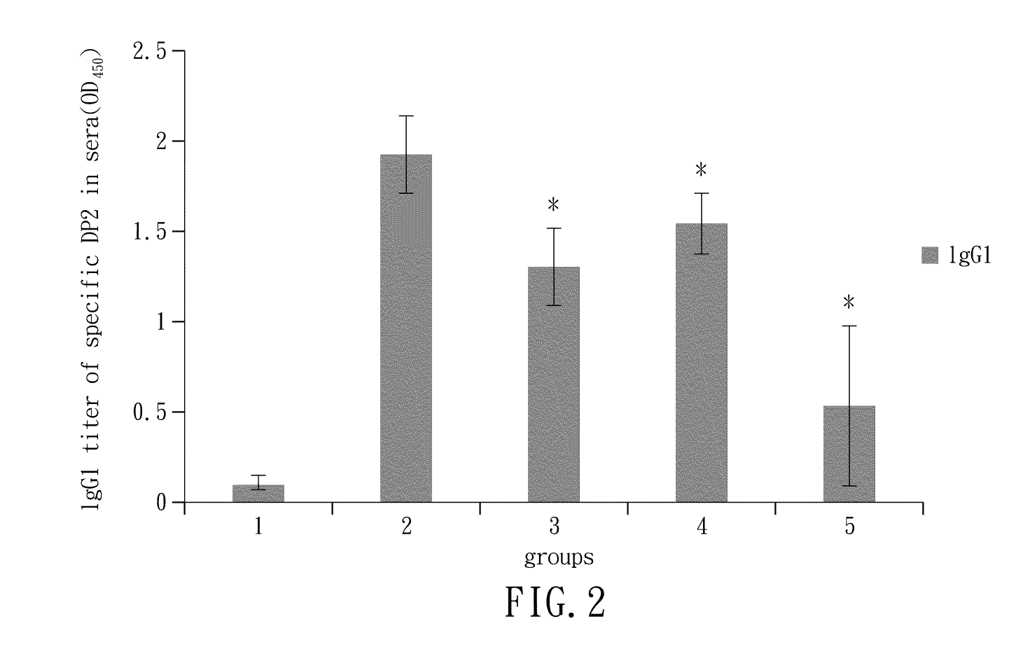

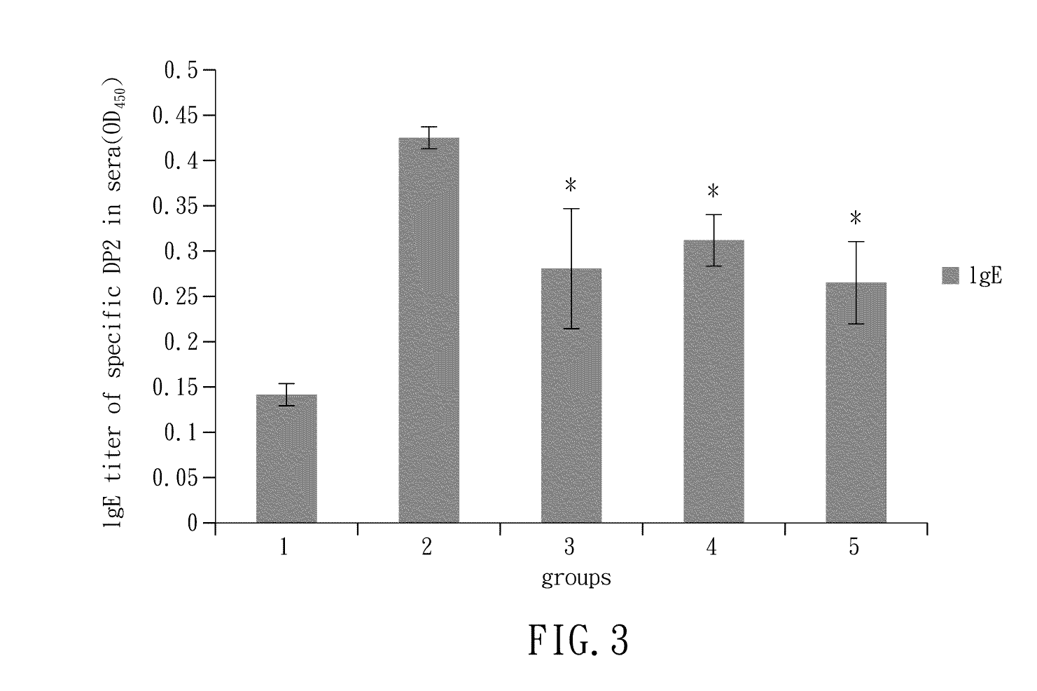

FIG. 2 shows that the level of IgG1 in sera in groups 4 and 5, i.e., mice treated with TCM-A and TCM-B, were significantly reduced as compared to that of group 2. The herbal composition, TCM-B (group 5), of this invention also demonstrated a superior effect in IgG1 reduction over dexamethasone. FIG. 3 shows that IgE levels in sera can be significantly reduced in groups 4 and 5. The above data reveal that the herbal composition of this invention is able to reduce Th2 antibodies, thereby alleviating allergic asthma.

### 4. Cytokine Evaluation

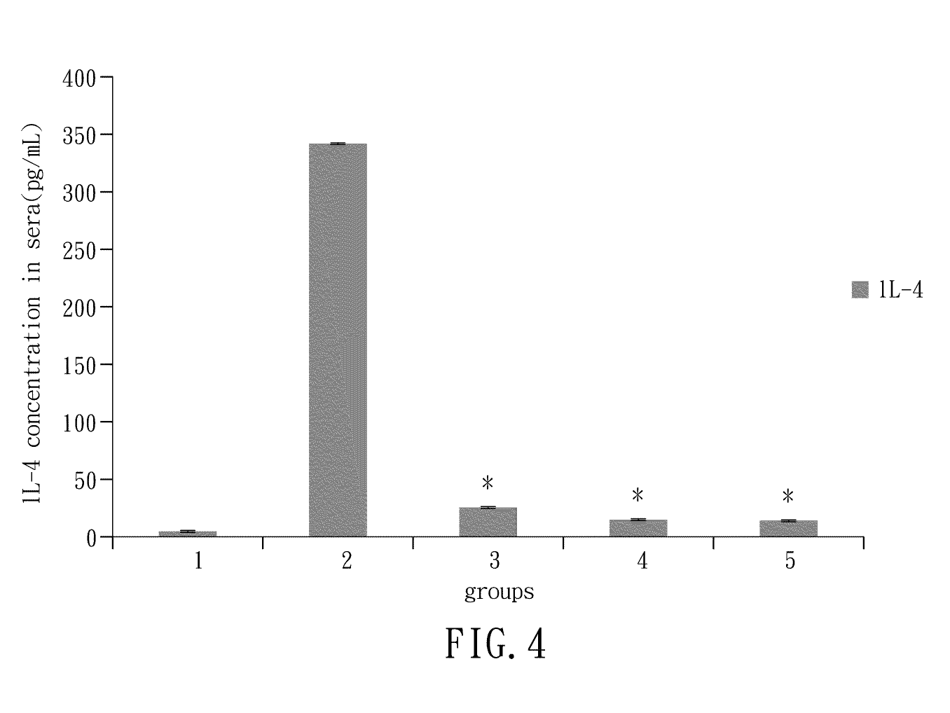

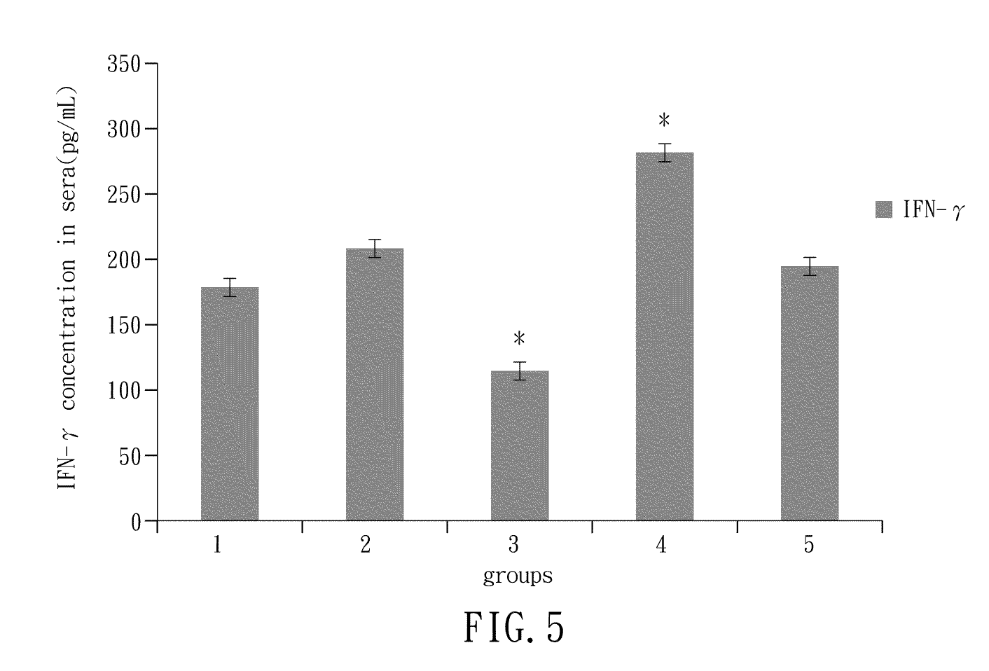

As shown in FIG. 4, the data reveals that IL-4, that is a Th2 cytokine, in groups 4 and 5, i.e., mice treated with TCM-A and TCM-B, were significantly reduced as compared to that of group 2. FIG. 5 shows that IFN-γ, that is effective in treating allergy, in group 4, i.e., mice treated with TCM-A, was significantly increased as compared to that of groups 2. The results demonstrate that the herbal composition of this invention is able to treat allergen-induced airway inflammation through cytokine regulation.

### 5. Histological Score

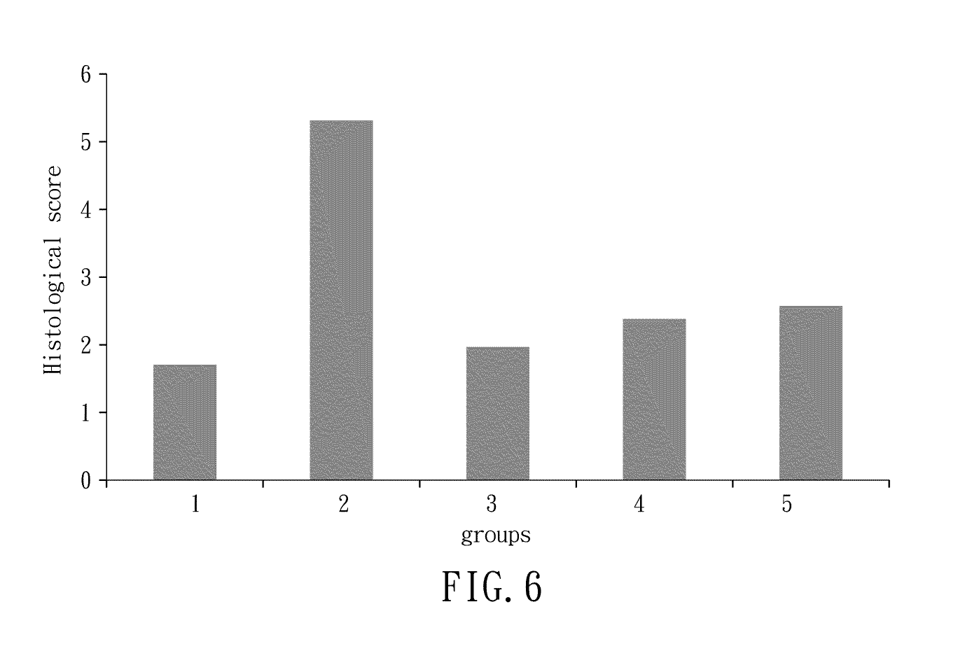

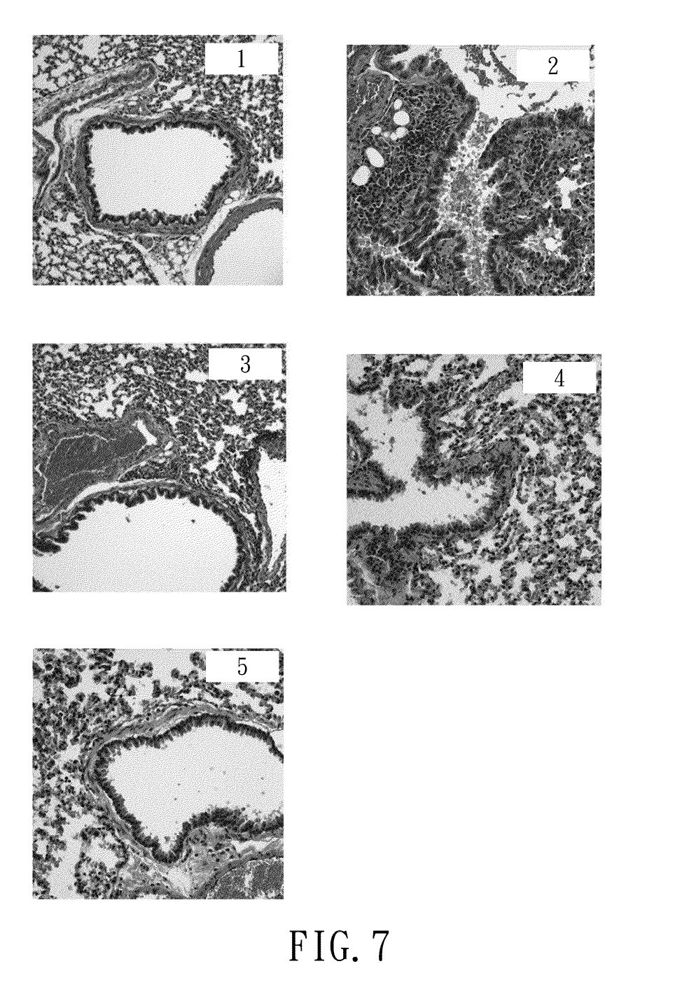

Histological evaluation was conducted based on the procedure set forth in “Histological evaluation” of section of “Materials and Methods”. Histological scores were determined according to Table 2. Lung tissues from each group of mice were evaluated and the results are shown in FIGS. 6 and 7. From FIG. 7, it reveals that, in group 2, mononuclear cells around blood vessels and around bronchioli were increased. Serious cell damage that causes tracheal rupture was found. However, in groups 3, 4, and 5, the number of mononuclear cell was reduced and cell damage situation was improved. Therefore, mice treated with Dexamethasone and the herbal composition of this invention both exhibited lower histological score, indicating that these treatments can reduce airway inflammation.

TABLE 2

| | Tracheal | | |
|---|---|---|---|
| Histological | thickness | Mononuclear | Tracheal |
| scores | (μm) | cell count | rupture |
| Level 1 | ≦25 | ≦5 | X |
| Level 2 | >25 and ≦35 | >5 and ≦15 | X |
| Level 3 | >35 and ≦40 | >15 and ≦20 | ◯ |
| Level 4 | >40 | >20 | ◯ |

In conclusion, the herbal composition of the present invention is novel and has similar and even superior effect in treating asthma as compared to dexamethasone. The herbal composition of this invention can successfully reduce asthmatic symptoms and increase lung function. The example suggests that the herbal composition of this invention is safe and very effective in improving pulmonary function to allergen, and may provide a good therapeutic means to patients with allergic asthma.

While the present invention has been described in connection with what are considered the most practical and preferred embodiments, it is understood that this invention is not limited to the disclosed embodiments but is intended to cover various arrangements included within the spirit and scope of the broadest interpretation and equivalent arrangements.

What is claimed is:

1. An herbal composition, comprising Dioscorea opposita, Nelumbo nucifera seeds, Euryale ferox seeds, Poria cocos, Diospyros kaki, Prunus dulcis, Mentha piperita leaves, and Panax quinquefolius.

2. The herbal composition of claim 1, further comprising Fritillaria unibracteata and Schizonepeta tenuifolia.

3. The herbal composition of claim 1, wherein Dioscorea opposita, Nelumbo nucifera seeds, Euryale ferox seeds, Poria cocos, Diospyros kaki, Prunus dulcis, Mentha piperita leaves, and Panax quinquefolius are in the ratio of 1:1:1:1:4:4:4:10 by weight.

4. The herbal composition of claim 2, wherein Fritillaria unibracteata, Dioscorea opposita, Nelumbo nucifera seeds, Euryale ferox seeds, Poria cocos, Diospyros kaki, Prunus dulcis, Mentha piperita leaves, Schizonepeta tenuifolia and Panax quinquefolius are in the ratio of 20:1:1:1:1:4:4:4:4:10 by weight.

5. The herbal composition of claim 1, further comprising a pharmaceutically acceptable carrier.

6. The herbal composition of claim 5, wherein the pharmaceutically acceptable carrier is selected from the group consisting of solvents, emulsifiers, suspending agents, disintegrators, binders, excipients, preservatives, lubricants, absorption delaying agents, liposomes, coloring agents, flavoring agents, and stabilizers.

7. A method for treatment of airway inflammation in a mammal, comprising administering to the mammal in need of such treatment an herbal composition, the herbal composition including Dioscorea opposita, Nelumbo nucifera seeds, Euryale ferox seeds, Poria cocos, Diospyros kaki, Prunus dulcis, Mentha piperita leaves, and Panax quinquefolius.

8. The method of claim 7, wherein herbal composition further includes Fritillaria unibracteata and Schizonepeta tenuifolia.

9. The method of claim 7, wherein, in the herbal composition, Dioscorea opposita, Nelumbo nucifera seeds, Euryale ferox seeds, Poria cocos, Diospyros kaki, Prunus dulcis, Mentha piperita leaves, and Panax quinquefolius are in the ratio of 1:1:1:1:4:4:4:10 by weight.

10. The method of claim 8, wherein, in the herbal composition, Fritillaria unibracteata, Dioscorea opposita, Nelumbo nucifera seeds, Euryale ferox seeds, Poria cocos, Diospyros kaki, Prunus dulcis, Mentha piperita leaves, Schizonepeta tenuifolia and Panax quinquefolius are in the ratio of 20:1:1:1:1:4:4:4:4:10 by weight.

11. The method of claim 7, wherein the herb composition further includes a pharmaceutically acceptable carrier.

12. The method of claim 11, wherein the pharmaceutically acceptable carrier is selected from the group consisting of solvents, emulsifiers, suspending agents, disintegrators, binders, excipients, preservatives, lubricants, absorption delaying agents, liposomes, coloring agents, flavoring agents, and stabilizers.

13. The method of claim 7, wherein the airway inflammation is allergic rhinitis, allergic conjunctivitis, or allergic bronchial asthma.

14. The method of claim 13, wherein the airway inflammation is allergic bronchial asthma.

15. The method of claim 7, wherein the mammal treated with the herbal composition has reduced IgG and IgE titers.

16. The method of claim 7, wherein the mammal treated with the herbal composition has decreased eosinophils cell number.

17. The method of claim 7, wherein the herbal composition is administrated to a mouse in a dose of 100 mg/kg/day to about 600 mg/kg/day.

18. The method of claim 17, wherein the herbal composition is administrated to a mouse in a dose of 500 mg/kg/day.
